(19) United States
(12) Patent Application Publication
(10) Pub. No.: US 2025/0009108 A1
(43) Pub. Date: Jan. 9, 2025

(54) Autonomous Skincare Routine Dispenser Leveraging Embedded Chip on Consumable

(71) Applicant: L'Oreal, Paris (FR)

(72) Inventors: Grégoire CHARRAUD, Clichy (FR); Matthieu JACOB, Clichy (FR)

(73) Assignee: L'Oreal, Paris (FR)

(21) Appl. No.: 18/712,598

(22) PCT Filed: Nov. 29, 2002
(86) PCT No.: PCT/EP2022/083713

§ 371 Date: May 22, 2024

(30) Foreign Application Priority Data
Nov. 30, 2021 (FR) 2112734

(51) Int. Cl. A45D 40/24; A45D 44/00; A61K 8/365; A61K 8/67; A61K 8/73; A61K 8/9789; A61Q 19/00

(52) U.S. Cl. CPC A45D 40/24; A45D 44/005; A61K 8/365; A61K 8/671; A61K 8/676; A61K 8/678; A61K 8/735; A61K 8/9789; A61Q 19/00; A45D 2044/007

(57) ABSTRACT

A dispenser is optionally connected to a computing device, and the computing device includes an app that is configured to specify the amounts of formulas to be dispensed by analyzing a user's skin or other factors to provide personalized formulas. The dispenser can also manage dispensing formulas without connecting to the computing device by reading an electronic communication and storage device on each cartridge containing a formula. The electronic communication and storage device of each cartridge stores data including one or more keys to check compatibility of the cartridges in the cartridge set, and one or more tracks, each track specifying dosages for the cartridge for each administering of formulas.

### CROSS-REFERENCE(S) TO RELATED APPLICATION(S)

This application claims the benefit of French Patent Application No. 2112734, filed on Nov. 30, 2021; the contents of which is hereby incorporated by reference in its entirety.

### SUMMARY

A system for enabling skincare and personalized formulas for use at home is based on a specialized dispenser that allows ingredients for a cosmetic product to be instantly blended into a user's preferred end result and then conveniently transported for portability. In one embodiment, the dispenser can be connected to a device, such as a smartphone, that allows managing the consumables through an app. The dispenser should be able to manage consumables when not connected to the app. This disclosure describes the management of the consumables, optionally without use of the app, to enable checking compatibility of cartridge sets, selecting recipes, checking expiration date, and controlling dosages. The management of consumables without use of the app is performed through an electronic communication and storage device residing on the consumables.

This disclosure provides leveraging the cartridges' electronic communication and storage device, such as RFID tags data format and device simple state machine. The dispenser is configured to read the data on the electronic communication and storage device and perform operations, such as consistence checks of the cartridge set, determining the priming status, changing recipes on a daily basis or chronologically. In one embodiment, the electronic communication and storage device is configured to use cyclic redundancy checking (CRC) and/or mathematic operations to ensure consistency of a cartridge set. In one embodiment, the use of encoded “keys” on the electronic communication and storage device can ensure the compatibility across cartridges. The cartridge consistency check can prevent mixing actives at dosages that should not mix. In one embodiment, the electronic communication and storage device includes hardcoded dispensing tracks or recipe tables describing the dosages in chronological order.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatical illustration of a system including a dispenser for dispensing formulations that can operate in a connected or offline manner;

FIG. 2 is a schematic diagram of the system of FIG. 1 showing the circuitry components of the system;

FIG. 3 is a diagrammatical illustration of a cartridge with an electronic communication and storage device;

FIG. 4 is a schematic diagram depicting the process of dispensing using the electronic communication and storage devices on cartridges;

FIG. 5 is a flow diagram depicting a method of dispensing using the electronic communication and storage devices on cartridges;

FIG. 6 is a state machine diagram of the dispenser using tracks stored on the electronic communication and storage devices on cartridges; and

FIGS. 7A to 7F are schematics of keys stored on the electronic communication and storage devices on cartridges showing examples of compatibility checking.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Referring now to the drawings, wherein like reference numerals designate identical or corresponding parts throughout the several views.

Selecting cosmetic and skincare formulations, and component materials to formulate cosmetic and skincare formulations, is a common activity often relying on subjective decision making and manual input. There are a wide variety of available materials, and countless combinations and permutations of possible cosmetic and skincare formulations.

For each occasion where cosmetic and skincare formulations are used, subjective decisions are often made by an end user to produce satisfactory formulations. Outcomes are generally the result of experimentation, perhaps requiring multiple iterations to produce a satisfactory outcome. Partly due to limited awareness of specific traits of the base materials and necessary proportions, resulting formulations may lack precision. The repeatability of producing a specific formulation is thus difficult to accomplish.

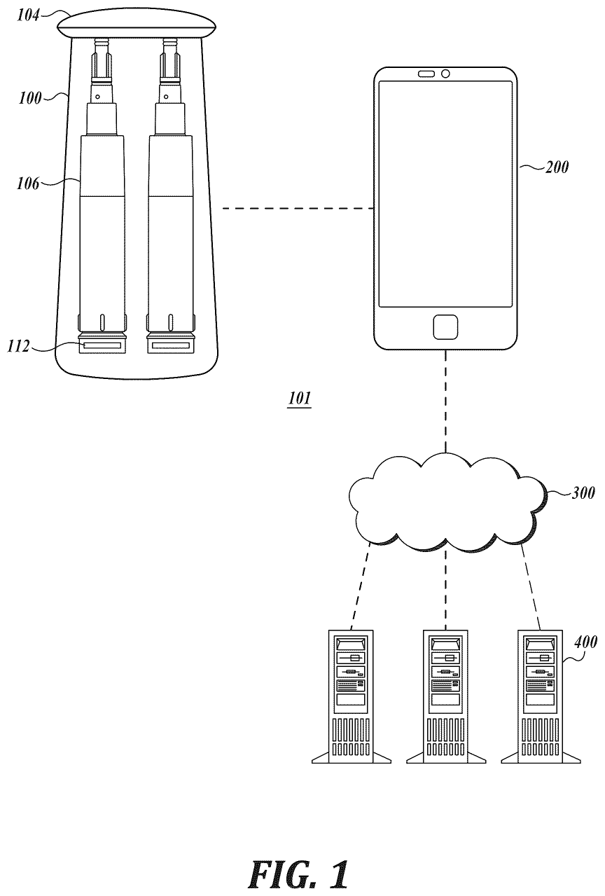

FIG. 1 relates to an eco-system 101 for enabling a cosmetic and skincare formula personalization system for use at home which is based on a specialized dispenser 100 that allows the ingredients for a formulation to be instantly blended into a user's preferred end result and then conveniently transported for portability.

The dispenser 100 includes a body containing two or more cartridges 106 and a compact 104 disposed above the dispenser body. The compact 104 can be a detachable mirrored compact to enable carrying the personalized formulation on the person.

Briefly described, the dispenser 100 includes a motor system, located on top, to compress the formula from the several cartridges 106 in the body of the dispenser in an upward motion to a receiving area, such as a dispensing tray or within the compact 104 above for a clean application. The formulas are dispensed in specified ratios to achieve a selected color or achieve a certain treatment. The formulation can be applied from the compact 104 while it is attached to the dispenser 100, or the compact 104 can be removed from the dispenser and carried on a person. The system shown can deliver personalized formulations, for skincare, haircare, foundation, lipstick, and others.

In one embodiment, the dispenser 100 is controlled from an app residing, for example, on a smartphone 200. The smartphone 200 can further connect to a server system 400 through the Internet 300.

In one embodiment, the app assesses users' individual skin and local environmental data to create and deliver personalized, on-the-spot skincare, haircare, and cosmetic formulas that optimize for increasing levels of personalization over time.

In one embodiment, the dispenser 100 is configured to operate partly or wholly offline and without connecting to the smartphone and without use of the app. The “autonomous” or “partly autonomous” operation of the dispenser 100 is based on data provided on electronic communication and storage devices 112 embedded within each of the cartridges 106. Examples of electronic communication and storage devices 112 suitable to be used on cartridges 106 include, but are not limited to, RFID chips, NFC tags, and the like.

When the dispenser 100 is used with the app, the overall eco-system features an AI-enabled, motorized cartridge system that creates personalized skincare and cosmetics formulas in four steps. The dispenser 100 creates personalized formulations through the following process when connected to a computing device, such as a smartphone with an app:
1. Personal skin analysis: The user takes a photo with a smartphone camera and opens an app on the smartphone. The app uses AI to analyze the user's overall skin condition, assessing deep wrinkles, fine lines, dark spots, lack of firmness, pore visibility, and lack of radiance.
2. Environmental assessment: The app (and/or a separate cloud computing platform) assesses local environmental conditions that can influence the state of the user's skin, including weather, temperature, humidity, UV index, air quality, and pollen.
3. Product preference: The user then enters specific skincare concerns, like fine lines, wrinkles, dark spots, rough skin texture and dullness into the app.
4. Custom formulation and dispensing: The app specifies the amounts of formulas and the dispenser dispenses a personalized blend of high-performance skincare formulas in a portioned, single dosage at the top of the device.

The operation of the dispenser 100 with the corresponding app have been described in previous publications, including U.S. 2021/0235845; U.S. 2021/0235849; U.S. 2021/0235850; U.S. 2021/0236390; and U.S. 2021/0236863, to which reference may be made.

When operating partly or wholly offline and without the app, data on the electronic communication and storage devices 112 on the cartridges 106 provide the instructions for operating the dispenser 100. In one embodiment, the electronic communication and storage devices 112 include data to operate the dispenser 100 as a “state machine” in order to dispense the formulas. In other words, the dispenser 100 is configured to dispense the formulas based on a user selected track or regimen and a chronologically ordered number of dosages. The selected track and a numbered dosage defines a state of the dispenser 100, for example. This allows the dispenser 100 to be controlled with hardcoded data on the electronic communication and storage devices 112.

In one embodiment, a dispensing counter is used to keep track of the chronological order of administering dosages. Dosages herein mean amounts of individual formulas dispensed from a cartridge. Incrementing the counter is an indication to the dispenser to move to the next chronological dosage for administration. However, even though the counter is incremented, dosages can remain the same for a given period of time. For example, similar dosages can be administered for a week before moving to a higher dosage. A track is programmed to include a finite number of dosages. The dispensing counter is used to step through all the number of dosages chronologically according to a preprogrammed order.

The dispensing counter is incremented by a trigger event. A trigger event can be any activity that indicates dispensing has occurred or will occur. In one embodiment, each time the compact 104 is sensed to be opened, the counter can increment. In one embodiment, each time the dispensing motor is sensed to be on, the counter can increment. However, other trigger events are possible, for example, a user can simply press a physical button on the dispenser 100 to active dispensing and the counter increments. In one embodiment, the trigger event is selected using a soft button on a touchscreen display 126 on the dispenser 100 after the dispenser display issues a message on the display and an audible signal.

In one embodiment, the electronic communication and storage devices 112 are coded with the tracks or personalization recipes after consulting with an advisor. For example, in a retail setting, a user may consult with an advisor at a store counter, spa, or the like. After the consultation, the advisor encodes the cartridge sets with personalized tracks for the user to use at home. In one embodiment, cartridge sets or individual cartridges encoded with tracks for wide application are sold to users.

In one embodiment, the electronic communication and storage devices 112 are preprogrammed with “keys” by the manufacturer. The keys allow the use of CRC (cyclic redundancy check) and mathematic operations to ensure consistency of a given set of cartridges and to ensure the compatibility of each cartridge with other cartridges in the set. The cartridge consistency check can also prevent mixing actives at dosages that should not mix.

One example of ingredients contained in cartridges 106 for skincare may include, alpha hydroxy acids (AHAs), Vitamins C and E, hyaluronic acid, ferulic acid, retinol, cucumber, thyme, and mulberry. However, other actives for skincare, haircare, and cosmetics may be used with the dispenser 100. Tracks are prepared for each cartridge having a different active. A track can be prepared for treating sensitive skin, for treating sun-exposed skin, and the like, the treatment of which may involve two or more of the actives. The track will include the total number doses to be administered and the dosage of each of the actives at each administration. Once the track and number of doses is determined, each cartridge, specifically the electronic communication and storage device 112 of each cartridge is encoded with the different tracks including the overall total number of doses and the dosages of each active corresponding to each track.

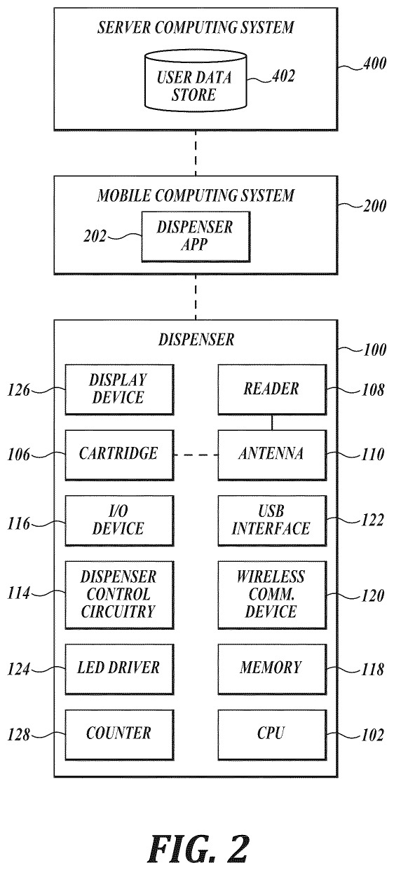

FIG. 2 is a schematic diagram representing an example of the optionally connected or optionally offline dispensing system 101. The system 101 which implements the dispenser 100 described above, includes at least the dispenser 100, and optionally a connected computing device 200, such as the smartphone. Optionally, the system 101 may further include one or more external servers 410 which are implemented as part of a cloud-computing environment communicating through the Internet 300.

In one embodiment, the dispenser 100 may operate without connecting to the computing device 200 and the app 202 and without being connected to the external servers 400 as described below.

Without connecting to the computing device 200 and without using the app 202, the dispenser 100 relies on the electronic communication and storage devices 112 in the cartridges 106 that may be inserted into the cosmetic device 100.

The connected computing device 200 may be a personal computer (PC), a laptop computer, a PDA (Personal Digital Assistants), a smartphone, a tablet device, a UMPC (Ultra Mobile Personal Computer), a net-book, or a notebook type personal computer.

The connected computing device 200 is capable of performing wireless communication with the dispenser 100 by way of a wireless communication interface circuitry 120 on the dispenser 100. However, connected computing device 200 is also capable of having a wired connection to the dispenser 100 by way of a USB interface 122 on the dispenser 100. Additionally, each device, including the dispenser 100, may communicate with each other and the external one or more devices through an internet connection via an 802.11 wireless connection to a wireless internet access point, or a physical connection to the internet access point, such as through an Ethernet interface. Each connected computing device 200 is capable of performing wireless communication with other devices, such as through a Bluetooth connection or other wireless means as well.

A server computing system 400 may be used. As shown, the server computing system 400 includes a user data store 402. “Data store” refers to any suitable device configured to store data for access by any one or more computing devices. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner on a computer-readable storage medium, such as a hard disk drive, a flash memory, RAM, ROM, or any other type of computer-readable storage medium. One of ordinary skill in the art will recognize that separate data stores described herein may be combined into a single data store, and/or a single data store described herein may be separated into multiple data stores. The connected device 200 may access the data store 402 when executing the app.

In a basic configuration, the connected device 200 includes at least one processor and a system memory connected by a communication bus. Depending on the exact configuration and type of device, the system memory may be volatile or nonvolatile memory, such as read only memory (“ROM”), random access memory (“RAM”), EEPROM, flash memory, or similar memory technology. The system memory typically stores data and/or program modules that are immediately accessible to and/or currently being operated on by the processor. In this regard, the processor may serve as a computational center of the computing device 200 by supporting the execution of instructions.

For case of illustration and because it is not important for an understanding of the claimed subject matter, FIG. 2 does not show some of the typical components of many computing devices. The connected computing device 200 may include a network interface comprising one or more components for communicating with other devices over a network. Embodiments of the present disclosure may access basic services that utilize the network interface to perform communications using common network protocols. The network interface may also include a wireless network interface configured to communicate via one or more wireless communication protocols, such as WiFi, 2G, 3G, LTE, WiMAX, Bluetooth, Bluetooth low energy, and/or the like.

The connected computing device 200 also includes a storage medium. However, services may be accessed using a computing device that does not include means for persisting data to a local storage medium. The storage medium may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

Suitable implementations of connected computing devices 200 that include a processor, system memory, communication bus, storage medium, and network interface are known and commercially available. The connected computing device 200 may include input devices, such as a keyboard, keypad, mouse, microphone, touch input device, touch screen, tablet, and/or the like. Such input devices may be coupled to the computing device 200 by wired or wireless connections including RF, infrared, serial, parallel, Bluetooth, Bluetooth low energy, USB, or other suitable connections protocols using wireless or physical connections. Similarly, the computing device 200 may also include output devices such as a display, speakers, printer, etc. Since these devices are well known in the art, they are not illustrated or described further herein.

The dispenser 100 includes a central processing unit (CPU) 102 which provides primary control over the separate circuitry components included in the dispenser 100, such as a dispenser control circuitry 114 (which may include control circuitry for the motors, circuitry for an optical encoder, and inductive sensor circuitry).

The CPU 102 may also control an optional input/output device 116 (such as a touchscreen, keyboard, or mouse), a memory 118, the wireless communication interface circuitry 120, the universal serial bus (USB) interface 122, an LED driver 124, a counter circuitry 128, and a display device 126. The LED driver 124 controls the pulsing of one or more indicator lights. The dispenser 100 includes circuitry for a reader 108 with antenna 110 for communicating with the electronic communication and storage devices 112 in the cartridges 106.

In one embodiment, the electronic communication and storage device 112 is a passive RFID tag. A passive RFID tag receives the energy to operate from an electromagnetic field that is produced by the reader 108. The reader 108 feeds an oscillating signal to the antenna 110. The signal is then rectified to charge a capacitor of the RFID tag. Data is exchanged between the electronic communication and storage devices 112 and the reader through the use of well-known signal modulation. In one embodiment, the electronic communication and storage devices 112 are read-only devices. In one embodiment, the electronic communication and storage devices 112 are read/write devices.

In one embodiment, circuitry includes, among other things, one or more computing devices such as a processor (e.g., a microprocessor, a quantum processor, qubit processor, etc.), a central processing unit (CPU), a digital signal processor (DSP), an application specific integrated circuit (ASIC), a field programmable gate array (FPGA), or the like, or any combinations thereof, and can include discrete digital or analog circuit elements or electronics, or combinations thereof.

In one embodiment, a module includes one or more ASICs having a plurality of predefined logic components.

In one embodiment, a module includes one or more FPGAs, each having a plurality of programmable logic components.

In one embodiment, circuitry includes one or more components operably coupled (e.g., communicatively, electromagnetically, magnetically, ultrasonically, optically, inductively, electrically, capacitively coupled, wirelessly coupled, or the like) to each other.

In one embodiment, circuitry includes one or more remotely located components.

In one embodiment, remotely located components are operably coupled, for example, via wireless communication, such as with the connected computing device 200.

In one embodiment, remotely located components are operably coupled, for example, via one or more communication modules, receivers, transmitters, transceivers, or the like.

In one embodiment, any of the CPU 102 or other circuitry components shown in FIG. 2 may be substituted with alternative circuitry elements. Examples of circuitry include memory that, for example, stores instructions or information. Non-limiting examples of memory include volatile memory (e.g., Random Access Memory (RAM), Dynamic Random Access Memory (DRAM), or the like), non-volatile memory (e.g., Read-Only Memory (ROM), Electrically Erasable Programmable Read-Only Memory (EEPROM), Compact Disc Read-Only Memory (CD-ROM), or the like), persistent memory, or the like. Further non-limiting examples of memory include Erasable Programmable Read-Only Memory (EPROM), flash memory, or the like.

In one embodiment, memory is coupled to, for example, one or more computing devices by one or more instructions, information, or power buses.

In one embodiment, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device.

In one embodiment, a module includes one or more user input/output components that are operably coupled to at least one computing device configured to control (electrical, electromechanical, software-implemented, firmware implemented, or other control, or combinations thereof) at least one parameter associated with, for example, determining one or more tissue thermal properties responsive to detected shifts in turn-ON voltage.

In one embodiment, circuitry includes a computer-readable media drive or memory slot that is configured to accept signal-bearing medium (e.g., computer-readable memory media, computer-readable recording media, or the like).

In one embodiment, a program for causing a system to execute any of the disclosed methods can be stored on, for example, a computer-readable recording medium, a signal-bearing medium, or the like. Non-limiting examples of signal-bearing media include a recordable type medium such as a magnetic tape, floppy disk, a hard disk drive, a Compact Disc (CD), a Digital Video Disk (DVD), Blu-Ray Disc, a digital tape, a computer memory, or the like, as well as transmission type medium such as a digital or an analog communication medium (e.g., a fiber optic cable, a waveguide, a wired communications link, a wireless communication link (e.g., receiver, transmitter, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

In one embodiment, circuitry includes acoustic transducers, electroacoustic transducers, electrochemical transducers, electromagnetic transducers, electromechanical transducers, electrostatic transducers, photoelectric transducers, radio-acoustic transducers, thermoelectric transducers, or ultrasonic transducers.

In one embodiment, circuitry includes electrical circuitry operably coupled with a transducer (e.g., an actuator, a motor, a piezoelectric crystal, a Micro Electro Mechanical System (MEMS), etc.).

In one embodiment, circuitry includes electrical circuitry having at least one discrete electrical circuit, electrical circuitry having at least one integrated circuit, or electrical circuitry having at least one application specific integrated circuit.

In one embodiment, circuitry includes electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs.

The dispenser 100 described above allows for swapping the consumable cartridges 106 in a smart and efficient manner. The cartridges 106 (consumables) used in the above-described dispenser 100 are preferably managed in sets (such as sets of three cartridges). For instance there could be separate sets of cartridges for each of the lipstick, skincare, haircare, and foundation applications described above. In the system, consumables sets are equipped with a smart chip or an electronic device configured to perform data storage and transmission/reception (such as NFC, RFID, or a contact chip). Each cartridge 106 has a different attributes and a unique formula identifier that can identify attributes such as Shade/finish, Texture, and Skin/hair benefits. Attributes are stored on the integrated circuit at production and signed with an asymmetrical cryptographic algorithm.

The dispenser can be operated offline and without the use the app. When operated offline, the electronic communication and storage device 112 applied to the cartridges 106 can be used to perform cyclic redundancy checks and mathematic operations to ensure consistency of a given set of cartridges and the compatibility of each cartridge with other cartridges, to control dosing schedules according to one or more dispensing tracks, and to monitor the cartridge priming operation.

Additionally, security mechanisms may implemented. Such security mechanisms can: (i) ensure the non-modification of production data: sector edition are protected by password (secret password); (ii) ensure the non-duplication of the cartridge data in case of diversion: adding a signature mechanism using MID (unique id of the tag, the data encoded, the secret key of the manufacture). The app using the device to read cartridge will then check that the signature comes from the manufacturing entity before allowing dispensing.

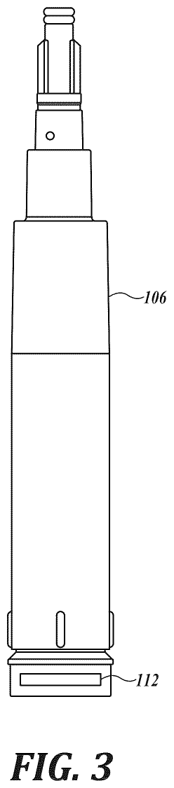

FIG. 3 shows a schematic of the cartridge 106, which includes the electronic communication and storage device 112 (smart chip, RFID, NFC tag, etc.) which is adhered to the bottom of the cartridge 106 in a manner such that it is flat and without edges. In one embodiment, data is encoded onto the electronic communication and storage device 112 using any generally known data format. For communicating with the cartridge's electronic communication and storage device 112, the dispenser 100 can includes a reader circuitry 108 and antenna 110 for each cartridge that can allow to read and write information onto the electronic communication and storage device 112 of each cartridge at each dispensing, for example.

The dispenser 100 and the connected computing device 200 may use a handshake process to start communications. The various triggers for initiating communication between the dispenser 100 and the connected computing device 200 may include a connection being established between the devices (such as a Bluetooth pairing), the lid of the dispenser being opened, a dispensing order from the app 202, or a dispensing order directly input on the dispenser 100. The handshake process can include, reading a consumable status of the cartridges 106 stored on the dispenser 100 and sending the status to the connected computing device 200. When not connected, status may be displayed on the display 126 of the dispenser 100.

In one embodiment, the dispenser 100 is operable without being connected to the computing device 200 or to the app 202. In one embodiment, the electronic communication and storage device 112 of each cartridge 106 includes data configured to allow operating the dispenser 100 when not connected to the computing device 200. In the offline or not connected state, the dispenser 100 communicates with the cartridges as described; however, the dispenser 100 may not be connected to the computing device 200. In one embodiment, the dispenser 100 may partly operate connected to the app and the computing device 200 initially, and thereafter operate without being connected to the computing device 200. In one embodiment, the dispenser 100 has the option to receive dosages from the connected computing device 200 wherein the dosages are determined by the app 202, or the dosages may optionally be selected based on the data stored on the electronic communication and storage devices 112 on the cartridges 106.

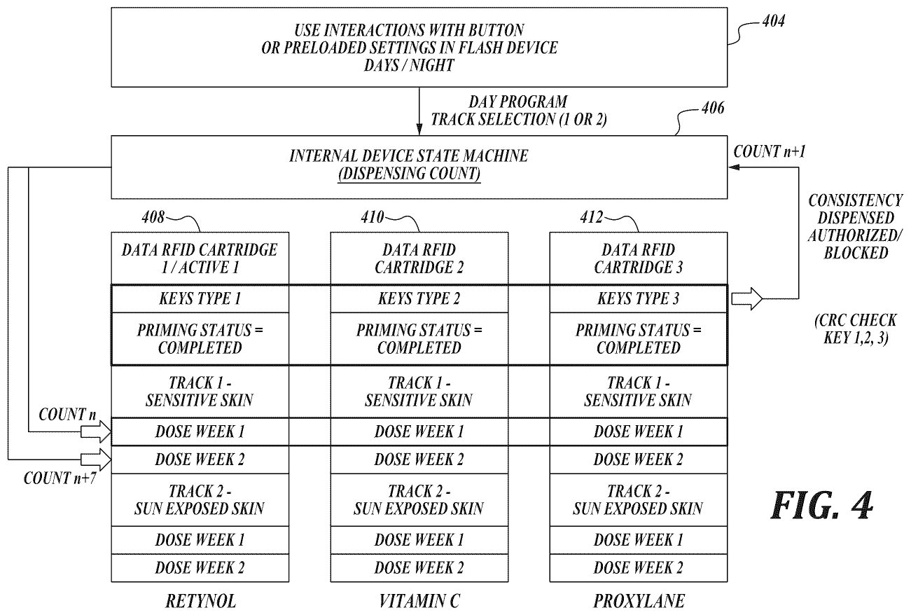

FIG. 4 shows a diagram depicting one example of operation of the dispenser 100 offline. In block 404, the user can initially interact with the dispenser 100 using a hard or soft button or with a flash device preloaded with settings for day or night schedules. In the illustration, a day program includes track 1 and track 2. The dosages are managed by considering the dispenser as a state machine based on the dispensing count.

Columns 408, 410, and 412 represent the data stored on each separate cartridge 106 on the electronic communication and storage device 112. In one embodiment, the data includes the keys for CRC, the priming status, and the tracks including the dosages.

Each cartridge has “keys” data that is used for checking compatibility of a cartridge set, using for example, CRC (cyclic redundancy checking). In one embodiment, the keys can be stored on NFC tags. A first key is designated “sub-type” describing the cartridge and a second “combination” key is used to allow introduction of new formulas and prevent two or more similar cartridges in a cartridge set. In one embodiment, the combination keys of the three cartridges must follow a rule to allow mixing of the formulas.

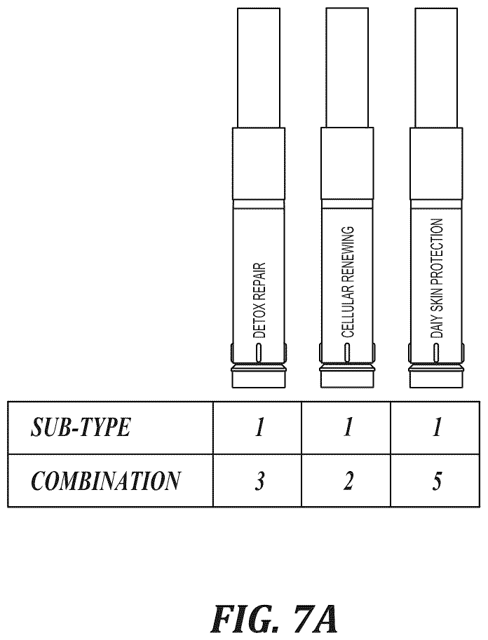

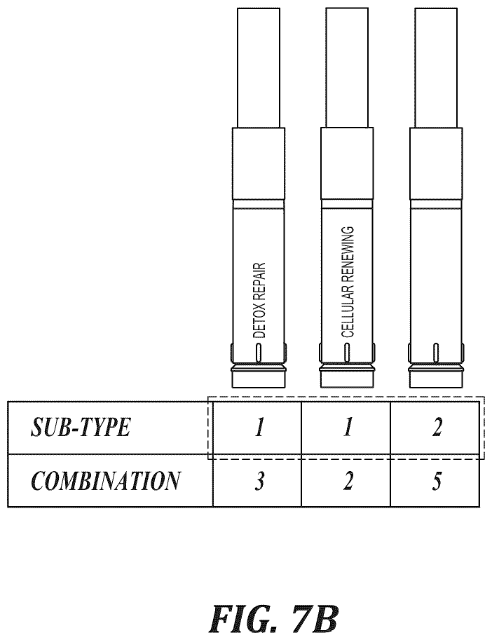

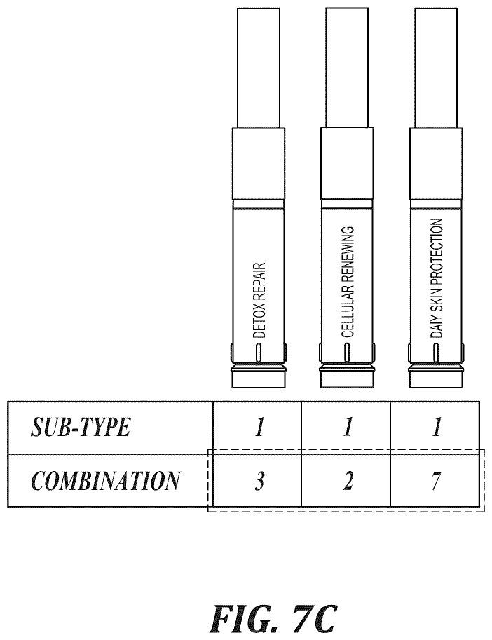

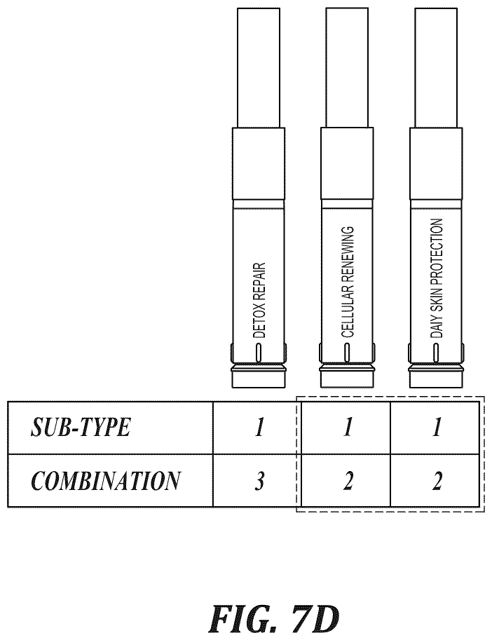

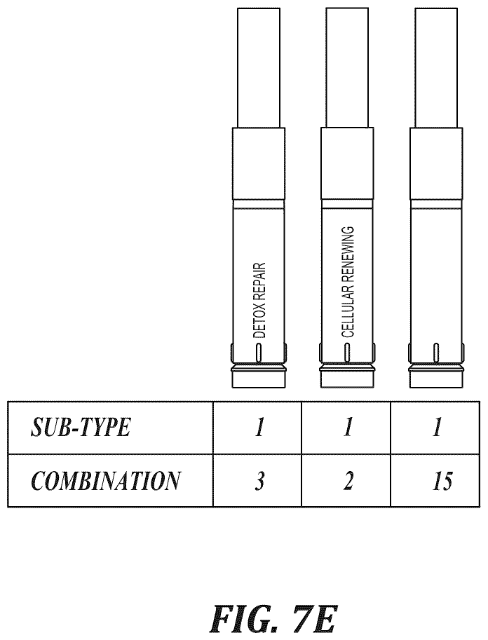

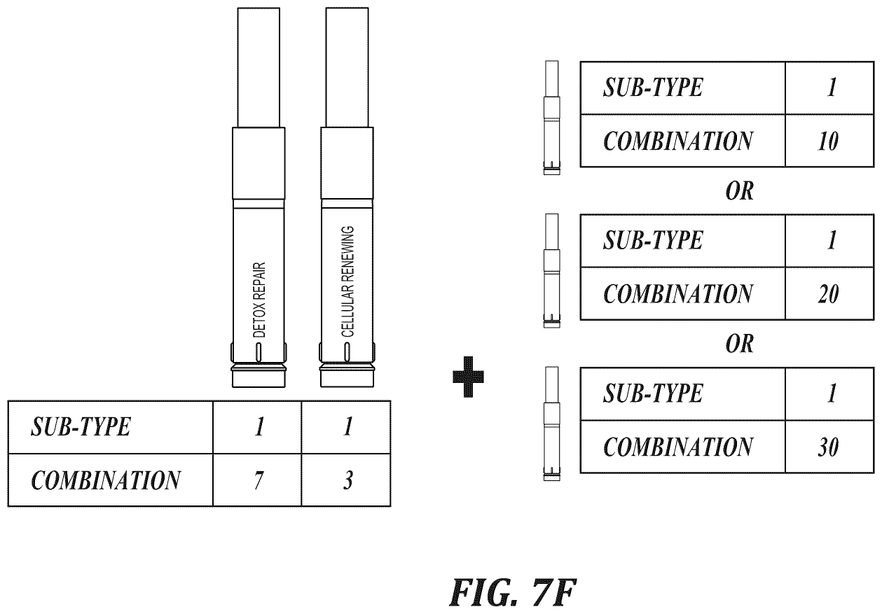

FIGS. 7A to 7F depict examples for checking that the cartridges in a cartridge set are compatible with each other and for allowing combinations of formulas to be mixed with each other. In the examples, three cartridges are assumed to be in a cartridge set.

In FIGS. 7A to 7F, keys data of three cartridges is represented numerically for ease of understanding. In the examples, three conditions are satisfied in order for the dispenser 100 to accept the cartridge set and allow dispensing. The first condition is that the sub-type key of the three cartridges is the same. The second condition requires that the combination key has to be different for each cartridge. This is to avoid having two or three of the same cartridge. The third condition requires that the combination keys respect a mathematical rule. For example, the sum of the three combination keys is a multiple of ten. This will allow introducing new formulas over time without impacting the dispenser firmware.

In FIG. 7A, it can be seen that the cartridges satisfy the first, second, and third conditions. Therefore, the cartridge set is accepted and dispensing is authorized by the dispenser 100. A pop-up message can be displayed on the dispenser display 126 indicating to the user that the cartridge set is accepted and further selections and information.

In FIG. 7B, it can be seen that while cartridges satisfy the second and third conditions, the first condition is not satisfied. Therefore, the cartridge set is not accepted and dispensing is blocked by the dispenser 100. A pop-up message can be displayed on the dispenser display 126 indicating to the user that the cartridge set is rejected and may include diagnostics that specifies the reason for rejection. The display 126 may include further selections.

In FIG. 7C, the first and second conditions are satisfied. The third condition is not satisfied. Therefore, the cartridge set is not accepted and dispensing is blocked. A pop-up message can be displayed on the dispenser display 126 indicating to the user that the cartridge set is rejected and may include diagnostics that specifies the reason for rejection. The display 126 may include further selections.

In FIG. 7D, the first and third conditions are satisfied. The second condition is not satisfied. Therefore, the cartridge set is not accepted and dispensing is blocked. A pop-up message can be displayed on the dispenser display 126 indicating to the user that the cartridge set is rejected and may include diagnostics that specifies the reason for rejection. The display 126 may include further selections.

In FIG. 7E, the first, second, and third conditions are satisfied. The cartridge set is accepted and dispensing is authorized. It is noted that a new cartridge is introduced where the sum of the combination is twenty. A pop-up message can be displayed on the dispenser display 126 indicating to the user that the cartridge set is accepted and further selections and information.

FIG. 7F is to illustrate that multiple additional cartridges can be added to the cartridge set as long as the newer cartridges have the same sub-type value, have a different combination value, and the combination value of the newer cartridge added to the combination values of the other two cartridges is a multiple of ten.

The use of NFC tags having sub-type and combination data, for example, allows for the management of validating cartridge sets without the use of the connected computing device 200.

Referring to FIG. 4, the electronic communication and storage device 112 may include the priming status of each of the cartridges 106. “Priming” refers to an initial process of making a cartridge ready for dispensing. Priming of cartridges 106 may be performed with use of the app 202 on the connected computing device 200. Once the priming of the cartridges 106 is completed, the priming status is written onto the electronic communication and storage device 112 corresponding to the cartridge 106. Thereafter, dispensing formula from a cartridge 106 that has been primed can be via the app 202 or without the app 202.

Briefly described, priming may include a step of dispensing some formula in a predetermined sequence and/or simultaneously from each of the cartridges to verify that dispensing can be performed from each cartridge. In an extra priming step, the user can practice clicking on a displayed color or formula to control individual dispensing on command. This may be performed to assure that the correct color is detected in the correct canal within the device such that recipes can be assigned automatically to the correct canal. When priming is complete, the status may be displayed on the display device 126 of the dispenser 100 or device 1002, and the priming status of the cartridges 106 is written onto the electronic communication and storage device 112. In one embodiment, once a cartridge is primed, the expiration date may be determined and tracked.

In some embodiments, priming of cartridges may be performed at a retailer, such as a counter at a store or at a spa. Once the cartridges 106 are primed and the status written to the electronic communication and storage device 112, the user may take the cartridges to be used in the home.

Referring to FIG. 4, the electronic communication and storage device 112 of each cartridge 106 further includes data relating to one or more tracks. A track describes a regimen for treating one condition. The illustrated embodiment includes a track for sensitive skin and a track for sun exposed skin. Each track contains the dosages for each of the three cartridges over a treatment period and the number of times a formula is dispensed.

In the illustrated embodiment, a first administered dosage of the formula from each cartridge is the same for each day during the first week of the track and a second administered dosage is the same for each day during the second week. It is understood that the length of the track, the number of dosages, and the time interval between the dosages can vary based on the track. It is also understood that the dosage amount and time interval between dosages can be different depending on the particular active being dispensed.

In one embodiment, the amount of the dosages are associated with a chronological order. The chronological order is determined by the counter 128 number. The counter 128 number corresponds to the number of times that dispensing has occurred.

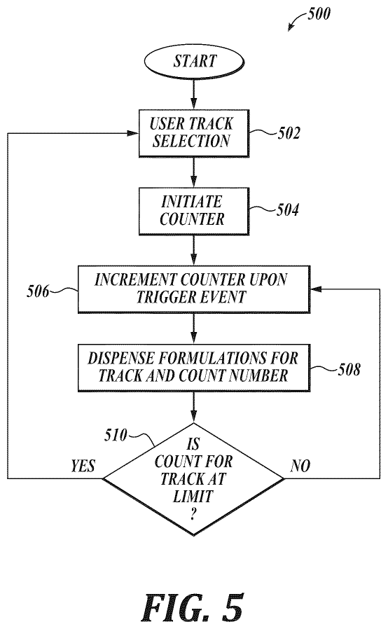

FIG. 5 is a flow diagram of a method for dispensing formulas from the dispenser 100 based on the track data on the electronic communication and storage device 112 when the dispenser 100 is not controlled by the app 202.

In block 502, the user selects the desired track. The display 126 on the dispenser 100 may show to the user the tracks that are available with the current set of cartridges 106. Each of the cartridges 106 will come pre-coded with the tracks in which the formula is used. The display 126 may also show other useful information, such as the priming status of each cartridge, the length of the track, the active formula in each cartridge, the remaining amount of formula in each cartridge, the time to go for a cartridge replacement, and other information that may be useful in the operation of the dispenser 100.

In block 504, the dispensing counter is initiated.

In block 506, the counter is incremented each time a dosage is dispensed. The trigger event to increment the counter can be any number of events, such as opening the compact, or depressing a hard or soft button to dispense the formula.

In block 508, the dispenser 100 dispenses the formulas corresponding to the track schedule and the counter number. The method of dispensing formulas based on track and in chronological order pre-coded onto the electronic communication and storage device 112 can be used for operating the dispenser 100 offline and without having to connect to the app 202.

In block 510, a determination is made whether the counter and therefore, the number of times a formula has been dispensed is at the end for the corresponding track. If the determination is “no,” indicating that dosages remain to be administered for the track, the dispenser waits until the next dosage is requested. If the determination is “yes,” indicating that the number of dosages have been completed for the track, the dispenser 100 waits for the user to select a new or the same track.

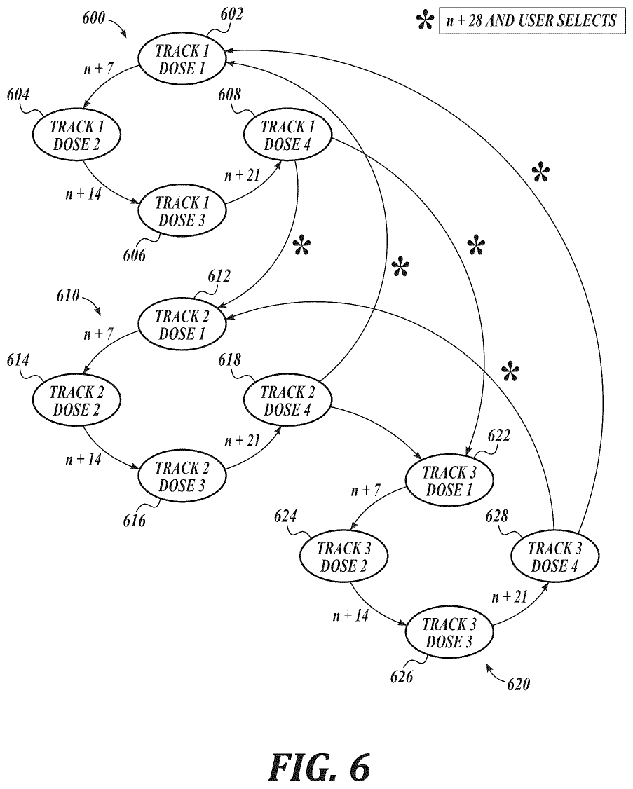

FIG. 6 is a machine state diagram for chronologically managing the administration of dosages of formulas from the dispenser 100 offline or without the app 202. To avoid obfuscating the features, a simplified illustration shows three tracks, wherein the same first dosage of each formula is administered in week 1, the same second dosage of each formula is administered in week 2, the same third dosage of each formula is administered in week 3, and the same fourth dosage of each formula is administered in week 4.

Track 600 includes four dosage levels, 602, 604, 606, and 608, each one being administered seven times, which may correspond to a daily dosage. Track 610 includes four dosage levels, 612, 614, 616, and 618, each one being administered seven times, which may correspond to a daily dosage. Track 620 includes four dosage levels, 622, 624, 626, and 628, each one being administered seven times, which may correspond to a daily dosage.

FIG. 6 illustrates the management of the dosages of formula when the dispenser 100 is not connected can be made to depend on a numerical counter that keeps count of the dispensing events chronologically. At the end of administering the predetermined number of dosages of any of the tracks, the conditions to move from one track to a different track require that the number of administering the fourth dosages has been completed, and that the user selects to begin a different track.

It is to be understood that FIG. 6 is only a representative example. The number of tracks can be more or less, the dosages of each cartridge do not have to be the same for each day, the dosages do not have to be administered once a day. Furthermore, each cartridge can have different dosages for each administration of formula. The illustrated example is made simple to show the basic principles.

While illustrative embodiments have been illustrated and described, it will be appreciated that various changes can be made therein without departing from the spirit and scope of the invention.

1. A system, comprising:
a dispenser configured to receive a cartridge set of two or more cartridges and to dispense a specified amount of formula from each cartridge, wherein each cartridge contains a formula and an electronic communication and storage device, wherein the electronic communication and storage device of each cartridge stores data including one or more keys to check compatibility of the cartridges in the cartridge set, and one or more tracks, each track specifying dosages for the cartridge for each administering of formulas.

2. The system of claim 1, wherein the dispenser includes a counter configured to increment upon a trigger event, wherein the counter keeps a chronological order, wherein each increment in the counter corresponds to each administering of formulas.

3. The system of claim 1, wherein the keys include a sub-type key and a combination key, and the dispenser includes a CPU configured to allow dispensing after performing a compatibility check using the keys.

4. The system of claim 3, wherein the sub-type key of each cartridge is the same, the combination key of each cartridge is different, and the combination keys of all cartridges follow a rule.

5. The system of claim 4, wherein the rule requires combination keys to be associated with a numerical value, and the sum of the numerical values is a multiple of a number determined in advance.

6. The system of claim 1, wherein each cartridge contains a skincare ingredient selected from the group consisting of alpha hydroxy acids, Vitamin C, Vitamin E, hyaluronic acid, ferulic acid, retinol, cucumber, thyme, mulberry, and combinations thereof.

7. The system of claim 1, wherein the electronic communication and storage device includes data indicating a status of each cartridge is ready for dispensing.

8. The system of claim 1, wherein the dispenser is optionally connected to a computing device, the computing device includes an app that is configured to perform a method including, analyzing a user's skin condition, assessing environmental conditions that influence a user's skin, receiving a skincare concern of a user, and specifying the amounts of formulas to be dispensed.

9. The system of claim 8, wherein the dispenser is configured to dispense formulas in dosages according to the method when connected to the computing device and in dosages according to the data stored in the electronic communication and storage devices when not connected to the computing device.

10. A method of managing dispensing of formulas from a dispenser including a cartridge set, comprising:
with the dispenser, reading an electronic communication and storage device on each cartridge containing a formula, wherein the electronic communication and storage device of each cartridge stores data including one or more keys to check compatibility of the cartridges in the cartridge set, and one or more tracks, each track specifying dosages for the cartridge for each administering of formulas.

11. The method of claim 10, wherein the dispenser includes a counter configured to increment upon a trigger event, wherein the counter keeps a chronological order, wherein each increment in the counter corresponds to each administering of formulas.

12. The method of claim 10, wherein the keys include a sub-type key and a combination key, and the dispenser includes a CPU configured to allow dispensing after performing a compatibility check using the keys.

13. The method of claim 12, wherein the sub-type key of each cartridge is the same, the combination key of each cartridge is different, and the combination keys of all cartridges follow a rule.

14. The method of claim 13, wherein the rule requires combination keys to be associated with a numerical value, and the sum of the numerical values is a multiple of a number determined in advance.

15. The method of claim 10, wherein the dispenser is connected to a computing device, the computing device includes an app that is configured to perform a method including, analyzing a user's skin condition, assessing environmental conditions that influence a user's skin, receiving a skincare concern of a user, and specifying the amounts of formulas to be dispensed, wherein the method is performed without connecting to the computing device, wherein each cartridge contains a skincare ingredient selected from the group consisting of alpha hydroxy acids, Vitamin C, Vitamin E, hyaluronic acid, ferulic acid, retinol, cucumber, thyme, mulberry, and combinations thereof.
